(19) United States
(12) Patent Application Publication
(10) Pub. No.: US 2008/0214947 A1
(43) Pub. Date: Sep. 4, 2008

(54) Exhaled Breath Condensate Collection and Assay System and Method

(76) Inventors: John F. Hunt, Charlottesville, VA (US); Brian K. Walsh, Amherst, VA (US); Alfred R. Baddour, Austin, TX (US); Dan Mackey, Esmont, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 11/660,653

(22) PCT Filed: Aug. 19, 2005
(86) PCT No.: PCT/US05/29604

§ 371 Date: Feb. 20, 2007

Related U.S. Application Data
(60) Provisional application No. 60/603,169, filed on Aug. 20, 2004.
(60) Provisional application No. 60/696,886, filed on Jul. 6, 2005.

(51) Int. Cl. A61B 5/00

(52) U.S. Cl. 600/532

(57) ABSTRACT

A method and system that provides for minute-to-minute EBC pH monitoring (or for other EBC characteristic monitoring) of a subject that greatly assists in determining the time-course of airway pH changes (or other characteristic changes) in evolving disease processes, and may assist in determining the predictive ability of these tests, as well as determining response to therapy.

### CROSS-REFERENCES TO RELATED APPLICATIONS

The present application claims priority from U.S. Provisional Patent Application Ser. No. 60/603,169, filed Aug. 20, 2004, entitled “Continuous Exhaled Breath Condensate Collection and Assay System and Related Method thereof,” and Ser. No. 60/696,886, filed Jul. 6, 2005, of which all of the disclosures are hereby incorporated by reference herein in their entirety.

The present application is also related to: U.S. Pat. No. 6,585,661 B1 issued Jul. 1, 2003, entitled “Device and Method for Monitoring Asthma;” U.S. Pat. No. 6,033,368 issued Mar. 7, 2000, entitled “Condensate Colorimetric Nitrogen Oxide Analyzer;” U.S. patent application Ser. No. 10/474,979, filed Oct. 16, 2003 (U.S. 2004/0127808 A1, published Jul. 1, 2004), entitled “Device and Method of Assessing Asthma and Other Diseases;” and U.S. patent application Ser. No. 10/257,912 (U.S. 2003/0208132 A1 published Nov. 6, 2003), filed Oct. 17, 2002, entitled “Method and Device for Collecting and Analyzing Exhaled Breath,” of which all of the disclosures are hereby incorporated by reference herein in their entirety.

### US GOVERNMENT RIGHTS

This invention was made with United States Government support under Grant No. FA9550-05-C-0012, awarded by the Air Force. The United States Government has certain rights in the invention.

### BACKGROUND OF THE INVENTION

Exhaled breath condensate (EBC) is a non-invasively collected body fluid that consists of expired condensed water and aerosolized particles of airway lining fluid, as well as volatile water soluble gases that are exhaled. Several hundred papers have now been published elucidating the potential of this fluid to supply knowledge regarding airway inflammation. Airway acidification has become increasingly recognized as contributing importantly to lung disease. A limitation in the art is that none of the respiratory monitoring systems or methods provides continuous, continual, semi-continual or semi-continuous collection of EBC and continual, continuous, semi-continuous or semi-continual monitoring and measuring thereof.

### SUMMARY OF THE INVENTION

The various embodiments of the present invention method and system provide for continuous, continual, semi-continual or semi-continuous collection of EBC and continual, continuous, semi-continuous or semi-continual monitoring and measuring thereof. For example, the various embodiments of the present invention method system and method may provide for minute-to-minute EBC pH monitoring (or for other EBC characteristic monitoring) which therefore would greatly assist in determining the time-course of airway pH changes (or other characteristic changes) in evolving disease processes, and may assist in determining the predictive ability of these tests, as well as determining response to therapy.

An aspect of an embodiment of the present invention method provides for monitoring a respiratory condition of a subject based on exhaled breath received from the subject. The method comprising: continuously or continually collecting exhaled breath condensate (EBC) produced from the subject's exhaled breath; and continually or continuously measuring one or more characteristics of the EBC. The method may further comprise gas standardizing the EBC.

An aspect of an embodiment of the present invention provides a method for monitoring a respiratory condition of a subject based on exhaled breath received from the subject. The method comprising: continuously or continually collecting exhaled breath condensate (EBC) produced from the subject's exhaled breath; and continually or continuously measuring pH of the EBC. The method may further comprise gas standardizing the EBC.

An aspect of an embodiment of the present invention provides a system for monitoring a respiratory condition of a subject. The system comprising: an exhaled breath port in communication with a condensate chamber; the condensate chamber being configured to condense the subject's exhaled breath received from the port and produce exhaled breath condensate (EBC); a first collection chamber configured to continuously or continually collect the EBC; a drain off port configured to drain the collected EBC in the first collection chamber as the collected EBC reaches a predetermined volume; and a means for continually or continuously measuring one or more characteristics of the collected EBC. The system may further comprise a gas standardizing means for standardizing the collected EBC from the subject

An aspect of an embodiment of the present invention provides a system for monitoring a respiratory condition of a subject. The system comprising: an exhaled breath port in communication with a condensate chamber; the condensate chamber being configured to condense the subject's exhaled breath received from the port and produce exhaled breath condensate (EBC); a first collection chamber configured to continuously collect the EBC; a second collection chamber in fluid communication with the first collection chamber and configured to continuously or continually collect the EBC from the first collection chamber as the first chamber collected EBC reaches a predetermined level in the first collection chamber; a drain off channel configured to drain the collected EBC in the second collection chamber as the collected EBC in the second collection chamber reaches a predetermined volume; and a means for continually or continuously measuring one or more characteristics of the collected EBC. The system may further comprise an inter-chamber port or other means configured to pass the collected EBC in the first chamber to the second chamber (and/or elsewhere as required or desired). The system may further comprise a gas standardizing means for standardizing the collected EBC from the patient. The gas standardizing means being in communication with the collected EBC in the first and/or second collection chamber (and/or elsewhere as required or desired).

These and other aspects of the disclosed technology and systems, along with their advantages and features, will be made more apparent from the description, drawings and claims that follow.

### BRIEF DESCRIPTION OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 is a schematic elevational view of an embodiment of a respiratory system.

FIG. 2 is a schematic elevational view of an embodiment of a respiratory system.

FIGS. 3(A)-(B) are schematic block diagrams of an embodiment of a respiratory system with respect to a subject.

FIGS. 4-8 represent the graphical data sets pertaining to experimental results obtained from select embodiments of the present invention.

### DETAILED DESCRIPTION OF THE DRAWINGS

Various systems, materials, and practices described herein, some of which constitute embodiments of the proprietary invention, provides a continuous, continual, semi-continual or semi-continuous EBC collection, deaeration and measurement system provided to determine the minute-to-minute changes in exhaled acid levels in diseases. Other time period changes may be practiced as desired or required, which may be greater or less than a minute. This same system for continuous, continual, semi-continual or semi-continuous collection can be used not only for monitoring pH (acid levels) but also for continuous or continual monitoring other constituents of EBC, including but not limited thereto, nitrogen oxides, redox potential, ammonia, conductivity, and any other dissolved constituents or characteristics of interest.

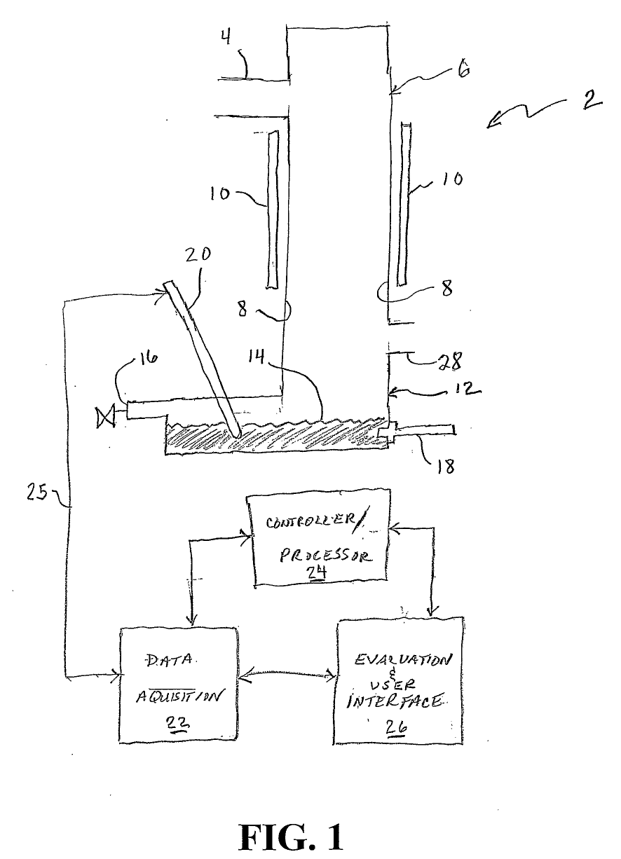

FIG. 1 is a schematic elevational view of an embodiment of a respiratory system 2 wherein exhaled breath from a subject is channeled or communicated through an exhaled breath port 4 into a condensate chamber 6. After the exhaled breath is channeled through exhaled breath port 4 the air condenses on the surface 8 of the condensate chamber 6 to form exhaled breath condensate (EBC). Some or all breath that is not condensed may exit through the air flow aperture 28. The condensate chamber 6 may be kept continuously, continually, semi-continually or semi-continuously chilled by a cooling device 10, such as cooling sleeve, ice, chiller, electric chiller, thermocouple systems, cold water bath, heat pump, heat sink, and/or endothermal chemical reaction. The cooling sleeve may comprise of aluminum or other material with desirable thermal conductive properties. In one embodiment, the chiller may be a commercially available water pumped system named Electri-Cool by Cincinnati Sub-Zero Products, Inc. As condensate forms on the condenser surface 8 condensate drips by gravity into a first collection chamber 12 to form EBC accumulation 14. Once the volume of the first collection chamber 12 is exceeded, as new EBC forms and enters the first collection chamber 12, the mixed EBC overflows through a drain off port 16. Alternatively, a sensor or the like (not shown) may determine when a desired volume threshold is achieved and drain off the EBC as desired. A gas or gas mixture supply 18 may be in communication with the EBC collected/accumulated in the first collection chamber 12 for purpose of, but not limited thereto, removing some or all carbon dioxide gas from the accumulated EBC 14. Alternatively, at desired times or instances the system 2 may be operated without using a gas or gas mixture supply. Similarly, the flow rate of gas and gas mixture supply may be controlled as well. This gas can be a variety of gases or gas mixtures, such as but not limited thereto the following: carbon dioxide filtered air, oxygen, nitrogen, argon, or any gas mixture from which carbon dioxide has been scrubbed or removed by commercially available means. For the hospital setting, oxygen may be preferred. Next a testing device 20 may be in communication with the accumulated EBC 14 to provide the continual, continuous, semi-continual or semi-continuous measurement of one or more characteristics of the EBC. The testing device 20 may be, for example an electronic monitor (such as but not limited to a pH probe or oxidation/reduction electrode), sensor device, or detector device or another probe. The testing device 20 is in communication with a data acquisition device 22. The data acquisition device 22 may also be in communication with a controller/processor 24 and evaluation & user interface module 26. The data acquisition device 22, controller/processor 24 and evaluation & user interface module 26 may be integral with one another or partially integral with one another or may be separate units as well. For example, the data acquisition device 22 and/or evaluation & user interface module 26 may provide continual, continuous, semi-continuous or semi-continual readings of the characteristic of interest of the EBC, such as pH, nitrogen oxides, oxidation/reduction potential (ORP), ammonia, conductivity, and/or a specific anion or cation probe, etc.

The testing device 20, data acquisition device 22, controller/processor 24 and/or evaluation & user interface module 26, or any combination thereof provides for the evaluation in determining the presence, absence or status of a disease or response to therapy. The disease, for example, may be a respiratory disease. Further, the evaluation may include determining presence, absence or status of at least one of the following: asthma, chronic obstructive pulmonary disease (COPD), ventilator-associated pneumonia (VAP), gastric acid reflux and aspiration, respiratory viral infections (including rhinovirus, respiratory syncitial virus, and others) chemical exposures (such as chlorine gas), near-drownings, acute respiratory distress syndrome (ARDS), acute lung injury, trauma (chest, multi-organ or other). The methods and systems discussed herein may assist in determining need for and response to therapies.

Alternatively, the exhaled breath port 4 may be located below the cooling device 10 and the air flow aperture 28 can be located above the cooling device 10. It should be appreciated that the flow aperture 28 and exhaled breath port 4, as well as the various components discussed throughout, may be located on various locations along the condensate chamber (or collection chamber), for example.

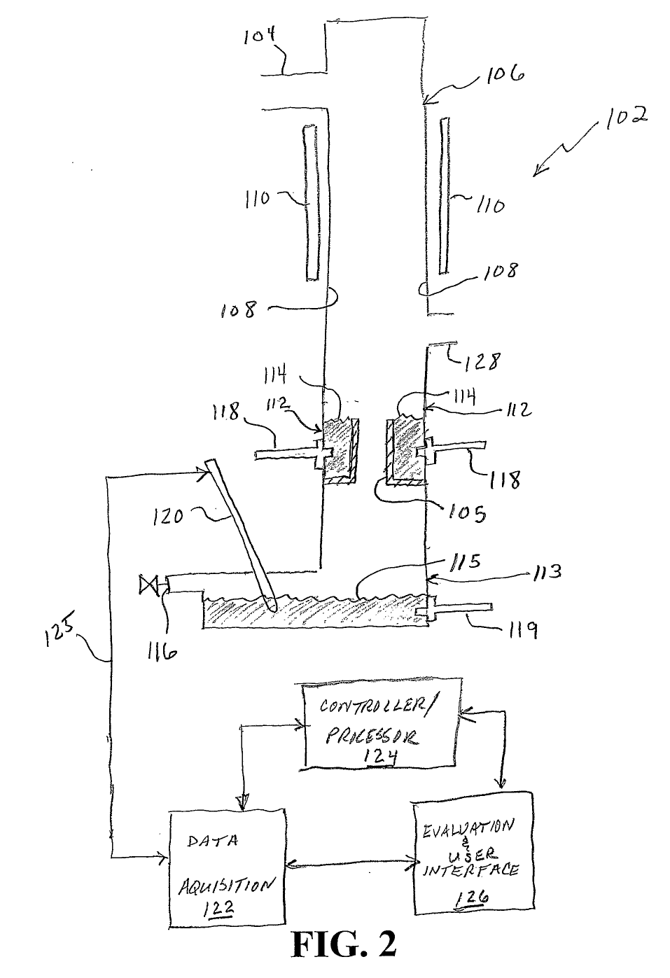

Turning to FIG. 2, FIG. 2 is a schematic elevational view of an embodiment of a respiratory system 102, similar to as shown in FIG. 1, wherein exhaled breath from a subject is channeled or communicated through an exhaled breath port 104 into a condensate chamber 106. After the exhaled breath is channeled through exhaled breath port 104 the air condenses on surface 108 of the condensate chamber 106 to form exhaled breath condensate (EBC). Some or all breath that is not condensed may exit through the air flow aperture 128. The condensate chamber 106 may be kept continuously, continually, semi-continually or semi-continuously chilled by a cooling device 110. As condensate forms on the condenser surface 106 condensate drips by gravity into a first collection chamber 112 to form an accumulation 114. Once the volume of the first collection chamber 112 is exceeded, as new EBC forms and enters the first collection chamber 112, the mixed EBC overflows through an inter-chamber port 105 into a second collection chamber 113. Alternatively, a sensor or the like (not shown) may determine when a desired volume threshold is achieved and allow the EBC to flow into the second chamber as desired (or alternatively to another location all together). It should be appreciated that the inter-chamber port 105 may be located on the inside of the chambers as illustrated or alternatively may be disposed on the walls of the chambers or outside the chambers. A gas or gas mixture supply 118 may be in communication with the EBC collected/accumulated in the first collection chamber 112 for purpose of, but not limited thereto, removing some or all carbon dioxide gas from the accumulated EBC 114. Similarly a gas or gas mixture supply 119 may in communication with the EBC collected/accumulated in the second collection chamber 113 for purpose of, but not limited thereto, removing some or all of the Carbon dioxide gas from the accumulated EBC 115. It should be appreciated that one or both of the gas supplies 118, 119 may be utilized or any combination thereof at different times of operation or volumes of accumulated EBC. Alternatively, at desired times or instances the system 102 may be operated without using a gas or gas mixture supply. Similarly, the flow rate of gas and gas mixture supply may be controlled as well. Once the volume of the second collection chamber 113 is exceeded or reaches a predetermined threshold (which may optionally be determined by a sensor), as new EBC forms and enters the first collection chamber 112 and/or second collection chamber 113, the mixed EBC overflows through a drain off port 116. Next a testing device 120 may be in communication with the accumulated EBC 115 to provide the continuous, continual, semi-continual or semi-continuous measurement of one or more characteristics of the EBC. The testing device 120 is in communication with a data acquisition device 122. The data acquisition device 122 may also be in communication with a controller/processor 124 and evaluation & user interface module 126. The data acquisition device 122, controller/processor 124 and evaluation & user interface module 126 may be integral with one another or partially integral with one another or may be separate units as well. For example, the data acquisition device 22 and/or evaluation & user interface module 26 may provide continual, continual, semi-continuous or semi-continual readings of the characteristic of interest of the EBC, such as pH, nitrogen oxides, redox potential, oxidation/reduction potential (ORP), ammonia, conductivity, and/or a specific anion or cation probe, etc.). It should be appreciated that the testing device 120 may be in communication with the accumulated EBC 115 of the second collection chamber 113 and/or the accumulated EBC 114 of the first collection chamber 112 (or may be utilized as any combination thereof at different times of operation or volumes of accumulated EBC) so as to provide the continuous, continual, semi-continual or semi-continuous measurement of one or more characteristics of the EBC.

The testing device 120, data acquisition device 122, controller/processor 124 and/or evaluation & user interface module 126, or any combination thereof provides for the evaluation in determining the presence, absence or status of a disease or response to therapy. The disease, for example, may be a respiratory disease. Further, the evaluation may include determining presence, absence or status of at least one of the following: asthma, chronic obstructive pulmonary disease (COPD), ventilator-associated pneumonia (VAP), gastric acid reflux and aspiration, respiratory viral infections (including rhinovirus, respiratory syncitial virus, and others) chemical exposures (such as chlorine gas), near-drownings injury/trauma, acute respiratory distress syndrome (ARDS), acute lung injury, trauma (chest, multi-organ or other). The methods and systems discussed herein may assist in determining need for and response to therapies.

It should be appreciated that there may be more than two collection chambers utilized and implemented (for example in series and/or parallel) for the present invention respiratory system according to the teachings and suggestions disclosed herein.

Alternatively, the exhaled breath port 104 may be located below the cooling device 110 and the air flow aperture 128 can be located above the cooling device 110. It should be appreciated that the flow aperture 128 and exhaled breath port 104, as well as the various components discussed throughout, may be located on various locations along the condensate chamber (or collection chambers), for example.

It should be appreciated that the communication of data and information transferred among the modules and components (data acquisition device 22, 122, controller/processor 24, 124, evaluation & user interface module 26, 126, and/or testing device 10, 110) of the respiratory system 2, 102 may be implemented using software and data transferred via communications interfaces that are in the form of signals, which may be electronic, electromagnetic, optical, RF, infrared or other signals capable of being received by communications interfaces. The signals may be provided via communications paths or channels 25, 125 (or any other communication means or channel disclosed herein or commercially available) that carries signals and may be implemented using wire or cable, fiber optics, integrated circuitry, a phone line, a cellular phone link, an RF link, an infrared link and other communications channels/means commercially available.

Examples evaluation & user interface module 26, 126 may include input devices, mouse devices, keyboards, monitors, printers or other computers and processors. The evaluation & user interface module 26, 126 (as well as the data acquisition device 22, 122) may be local or remote. It should be appreciated that there may be one or more evaluation & user interface module 26, 126 (as well as the data acquisition device 22, 122) that may be in communication with any of the components, modules, instruments, devices, systems and equipment discussed herein. For example, the evaluation & user interface module 26, 126 may be remotely located. Such a remote communication of the evaluation & user interface module 26, 126 may be accomplished a number of way including an uplink/communication path to a cell telephone network (e.g., external device/system) or satellite (e.g., external device/system) to exchange data with a central processing point (e.g., external device/system).

The controller/processor 24, 124 may be a variety of processors implemented using hardware, software or a combination thereof and may be implemented in one or more computer systems or other processing systems, such as general purpose computer or personal digital assistants (PDAs).

The data acquisition device 22, 122 may be a variety of meters, programmable logic controllers (PLC), and/or processors—as well as voltage/millivoltage, resistance, or conductivity meters with internal or programmable logic to convert voltage or other electrical measure into the output such as pH, conductivity, or given ion of interest—implemented using hardware, software or a combination thereof.

The data acquisition device 22, 122, controller/processor 24, 124 and/or evaluation & user interface module 26, 126 may be configured to be evaluating, monitoring and archiving storage of pH data or other characteristic data. The evaluation & user interface module 26, 126 (and/or data acquisition device 22, 122) may be configured to provide a user, technician, medical personnel with a touch screen graphical user interface for real-time monitoring and historical tracking and review of EBC pH values (or other characteristic values). Touchscreen controlled software controls may allow the user to initiate or monitor some of the following functions: calibration of pH probe (or testing device); start sampling of pH data (or other characteristic data); stop data sampling; save data sample set (externally or internally); and control appearance of graphical data display, including magnification of pH line (or characteristic line) from one minute of data per screen to 96 hours per screen (or other time parameters as desired).

In an embodiment, for example, measurement of pH (or other characteristic) using an appropriate electrode then provides a continuous or continual moving average of the EBC pH (or other characteristic). The smaller the volume of the chamber (first and/or second collection chamber) the shorter the time that is averaged into the assay. This allows production of a one minute to a sixty minute (or longer or shorter) continuous or continual moving average value (for example for pH). It should be appreciated that other mathematical calculations or recordation may be practiced rather than moving averages, such as recording individual data points and other statistical applications.

In some embodiments, for example, the present invention method and system may be used for, among other things, continuous, continual, semi-continual or semi-continuous exhaled breath condensate collection and assay for use in endotracheally intubated patients, tracheostomy patients, and for spontaneously breathing patients wearing facemasks such as are used for provision of oxygen, CPAP (continuous positive airway pressure), or BiPAP (BiLevel Positive Airway Pressure). The system can be incorporated directly into the expiratory limb of a ventilator circuit, but preferentially is located distal to the exhaust port of the ventilator. In an embodiment, the present invention system may be attached to a Servo I ventilator exhaust port.

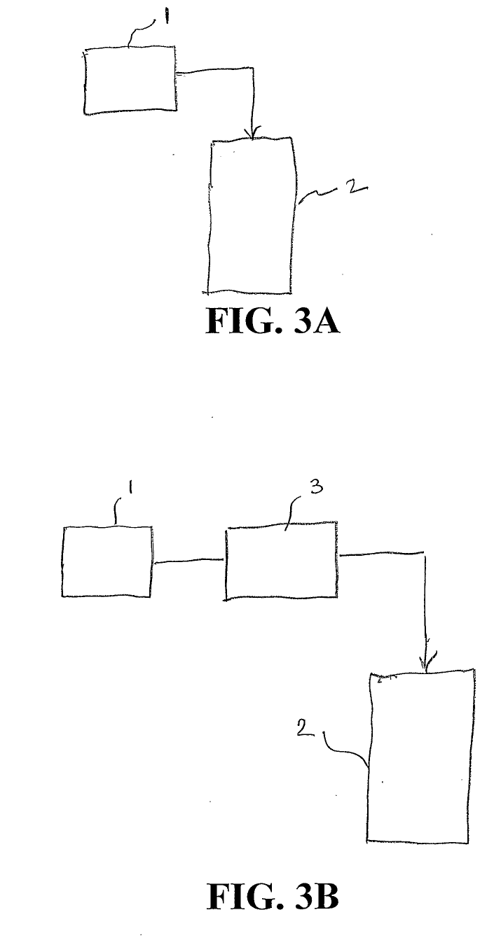

For instance, referring to FIG. 3(A), the subject 1 may be in direct communication with the present invention respiratory device 2 (or 102 as shown in FIG. 2) wherein the exhaled breath enters the exhaled port (4 or 104 as shown in FIGS. 1-2) of the device 2 (or 102 as shown in FIG. 2). Alternatively, referring to FIG. 3(B), the subject 1 may be in indirect communication with the present invention respiratory device 2 (or 102 as shown in FIG. 2) wherein the exhaled breath from the patient enters an invasive or non-invasive device 3 before traveling to the exhaled port (4 or 104 as shown in FIGS. 1-2) of the device 2 (or 102 as shown in FIG. 2). Examples of non-invasive devices include, but not limited thereto, the following: mask, mouthpiece, or other available non-invasive breath collection or directing systems. The non-invasive devices may be positive airway pressure (PAP), continuous positive airway pressure (CPAP) or Bilevel positive airway pressure (BiPAP). Examples of invasive devices include, but not limited thereto, the following: endotracheal device, endotracheal tube, tracheostomy tube or other available invasive breath collection or directing systems. The invasive devices may be PAP, CPAP, IPPV (intermittent positive pressure ventilation), SIMV (synchronized intermittent mandatory ventilation) or BiPAP, or mechanical machines used commonly in intensive care units, operating rooms or at home.

The subject 1 may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have respiratory characteristics similar to human (e.g., cow). It should be appreciated that the subject 1 may be any applicable patient, for example.

In some embodiments, for example, the present invention method and system may be used with, among other things, for continuous, continual, semi-continual or semi-continuous moving average assays. Thus the first and/or second collection chamber sizes will reflect the exhaled breath collected in the previous minute, five minutes, twenty minutes or hour (etc.) depending on the collection chamber size chosen and amount of EBC collected per minute. For instance, in a particular embodiment a ten minute moving average of EBC pH would thus be provided with a chamber size of about threes cc's and a rate of EBC production of about three cc's per ten minutes. New EBC formed is rapidly mixed into the first and/or second collection chambers and displaces previously collected EBC from the first and/or second collection chambers. Thus the sample is constantly refreshed to reflect the most recent happenings in the subject's lungs. The rate, number of repetitions, and duration of breathing, collection and assaying/testing/measuring may be modified according to desired and required applications and relationship with breathing, collection and/or assaying/testing/measuring.

In some embodiments, for example, the present invention method and system may be used for, among other things, continuous, continual, semi-continual or semi-continuous non-invasive monitoring of pathologically relevant deviations in lung chemistry (such as airway pH) that can be accomplished with robust and simple methodology in patients on mechanical ventilators. Minute to minute (or other designated time spans) breath acid levels can be assayed immediately and automatically, providing a continuous or continual output and charting of the breath acid levels. The present invention method and device can monitor nitrogen oxides, redox potential and other important aspects of lung chemistry and inflammation. The present invention method and system does not interfere with any aspect of ventilator functioning or monitoring ability. The embodiments of the present invention methodology will allow rapid growth in our understanding of airway chemistry and inflammation and how they change in disease, and has potential to become a standard monitoring tool in the intensive care setting for all patients on ventilators, equivalent to oxygen saturation monitoring.

In some embodiments, for example, the present invention method and system may be used for, among other things, hospitals (e.g., intensive care, operating rooms, and therapeutic facility), sleep labs, clinics, out patient surgery, ventilator manufacturers, home kits, shipboards, battalion aid stations, medical treatment facilities, and/or pharmaceutical companies.

In some embodiments, for example, the present invention method and system may be used as an intentionally redundant sample overflow system. This may be present to prevent accumulation of EBC in the first and/or second collection chambers and up the device to the point where it could potentially drain back proximally into the exhaust port of the ventilator or through the exhaled breath port or air flow aperture of the device. This is primarily a safety issue, but secondarily has relevance because the nature of this system controls the volume of EBC in the first and/or second collection chambers, and that volume(s) determines approximately the interval of the moving average pH reading. The EBC condensation may continues at a steady pace from hour to hour, for example, unless the patient demand changes, the ventilator settings are substantially adjusted, or the ventilator circuit is interrupted. The collected EBC is directed by gravity into the first collection chamber and then possible into the second collection chamber which also serves as the pH assay chamber (possibly in addition to the first chamber). The volume of EBC that is allowed to stay in this second collection chamber will determine the moving average interval. Thus if the fluid volume in this second collection chamber is maintained at one ml, and the EBC accumulation rate is six ml/hour, the EBC pH reading will provide a ten minute moving average, reflecting the EBC pH average over the previous ten minutes. As new EBC enters these chambers, the new EBC partially displaces the older EBC, and if this new EBC is more acidic, the pH change is noted. It should be appreciated that this situation can fluctuate in that the rate of EBC accumulation may be higher or lower than six ml/hour. If higher, than the moving average becomes tighter (for example a five minute moving average). If lower volumes of EBC are collected, as occurs in smaller patients, then the moving average is longer (for example, twenty minutes). Optimally, therefore, the volume of fluid allowed in the pH assay chamber should be adjustable depending on the volume of EBC accumulating. In practicality, the collected EBC volume will depend on the exhaled volume from the patient plus the additional bias flow gas, as well as the condenser efficiency.

In some embodiments, for example, the present invention method and system may be used with a condensation chamber 6, 106, that will be constructed from high heat conductivity materials. For example, the material may be aluminum as aluminum is reasonably inexpensive, lightweight and transportable, has high thermal conductivity, and is easily machinable to diverse geometries. The surface condensation chamber 6, 106, may be coated with a baked-on TEFLON coating which is hydrophic, repelling accumulated EBC off the surface readily as it accumulates, allowing for faster movement of EBC from the condenser surface into the collection chambers.

Moreover, it should be appreciated that the various components of the respiratory device may be a variety of commercially available materials used for respiratory device/systems. Some examples of materials used for the condensate chamber and collection chambers (as well as other components of the present invention system) may include, but not limited thereto, the following: polymers, rubber, plastic, polypropylene, composites, metals, ceramics, hydrogels, dialysis membranes, alloys, and other membranous materials, and other organic and inorganic compounds and substances and the like. It should be appreciated that the various components of the present invention system 2, 102, including its components, may be flexible or rigid and combination thereof as required or desired for intended use. Similarly, the system 2, 102 including its components, may provide volume contoured respiration by adjusting its geometry and flexibility/rigidity according to the subject location or anatomy being treated.

It should be appreciated that the condensate chamber and collection chambers may be comprised of a variety structures including, but not limited thereto, the following: constituting various types of conduits, tubes, hoses, channels, passages, pipes, tunnels, and/or bounded tubular surfaces or the like. Moreover, the condensate chamber and collection chambers may have a variety of cross-sectional shapes including, but not limited to the following geometric shapes: circular, oval, multi-faceted, square, rectangular, hexagonal, octagons, parallelogram hexagonal, triangular, ellipsoidal, pentagonal, octagonal, or combinations thereof or other desired shapes, including variable diameter or cross-section geometries and irregular geometries.

Further, it should be appreciated that any of the ports and/or apertures discussed herein may have a variety of shapes such as, but not limited thereto, the following circular, oval, multi-faceted, square, rectangular, hexagonal, octagons, parallelogram hexagonal, triangular, ellipsoidal, pentagonal, octagonal, or combinations thereof or other desired shapes.

Similarly, the ports or apertures discussed herein may be of a variety structures such as, but not limited thereto, the following: recess, port, duct, trough, tubes, hoses, bore, inlet, hole, perforation, channel, passage, slot, orifice or the like.

It should be appreciated that the duration of the continuous or continual collections may be less than the durations of a subject's exhaled breath, equal to the duration of an exhaled breath, and/or greater than the duration of an exhaled breath. Further, any continuous or continual collection may comprise one or more interruptions. Additionally, any continual or continuous measurement may have a duration that is at least as great as a period or instance sufficient to acquire data, digital data or electrical measure required for such measurement. Such acquisition of data may be accomplished using an electronic, electrical or analog device discussed throughout or any combination thereof.

Moreover, it should be appreciated that the duration of the continuous or continual collections may be any period necessary for the patient to receive treatment, therapy, participate in any experiment/study/test or undergo monitoring. Such continuous or continual collections may comprise one or more interruptions. An example may be that a hospital patient may receive treatment on ventilator for hours, days or even years. An additional example may be that a patient may be subjected to respiratory monitoring for sleep apnea or other diseases, or undergoes invasive or non-invasive positive pressure ventilation for sleep apnea or other diseases, whereby the collection takes place over period of hours or as required. The duration of the continuous or continual collections, which may include one or more interruptions, may include a variety of time ranges including, but not limited thereto, the following: less than about 1 minute; about 0.5 minutes to about 5 minutes; about 1 minute to about 1 hour; about 1 hour to about 12 hours; about 1 day to about 1 year; and/or greater than about 1 year, as well as any desired period as determined by the clinician, user or technician.

### EXAMPLES

Aspects of the various embodiments of the present invention are now described with reference to the following examples. These examples are provided for the purpose of illustration only and the invention should in no way be construed as being limited to these examples, but rather should be construed to encompass any and all variations which become evident as a result of the teachings provided herein.

### Example No. 1

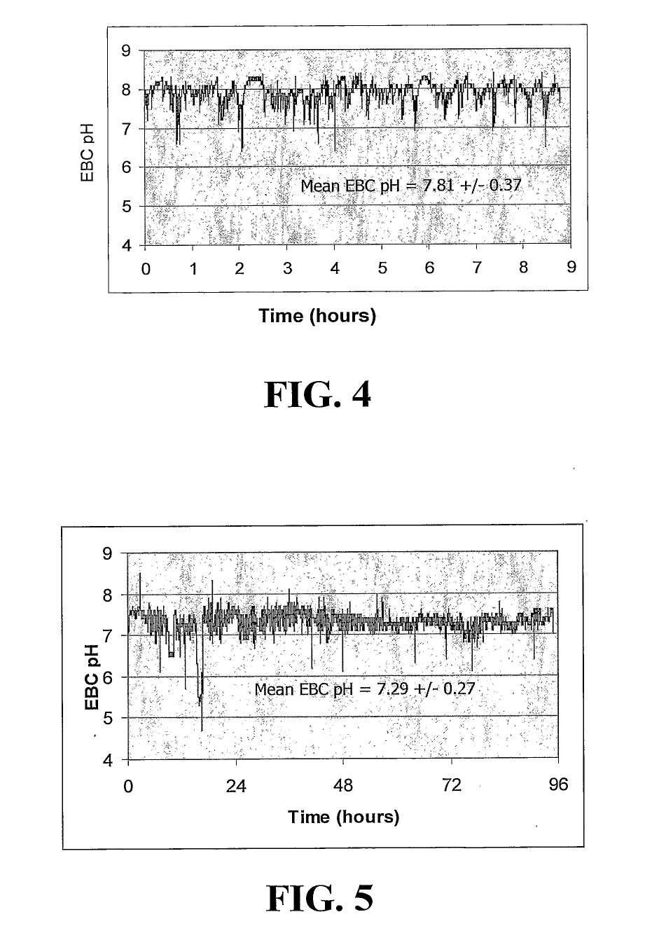

Referring to FIG. 4, FIG. 4 represents the graphical data sets captured during continuous or continual nine hour EBC pH tracing in a patient intubated post trauma, without lung injury. Note the baseline EBC pH of approximately 7.8-8.0, which is identical to normal values obtained in isolated collections from spontaneously breathing patients. Also note the frequent transient declines in EBC pH. These are possibly acid reflux and aspiration events, despite the presence of an endotracheal tube.

### Example No. 2

Referring to FIG. 5, FIG. 5 represents the graphical data sets captured from a 3-year-old patient with cystic fibrosis, stable for several weeks on ventilation for liver failure and prominent bronchiectasis, whereby 96 hours of continuous or continual EBC tracing was conducted. Note the generally lower baseline EBC pH than the trauma patient shown in FIG. 4. This low baseline is consistent with oral collections in cystic fibrosis patients, whom generally have a lower than normal EBC pH.

### Example No. 3

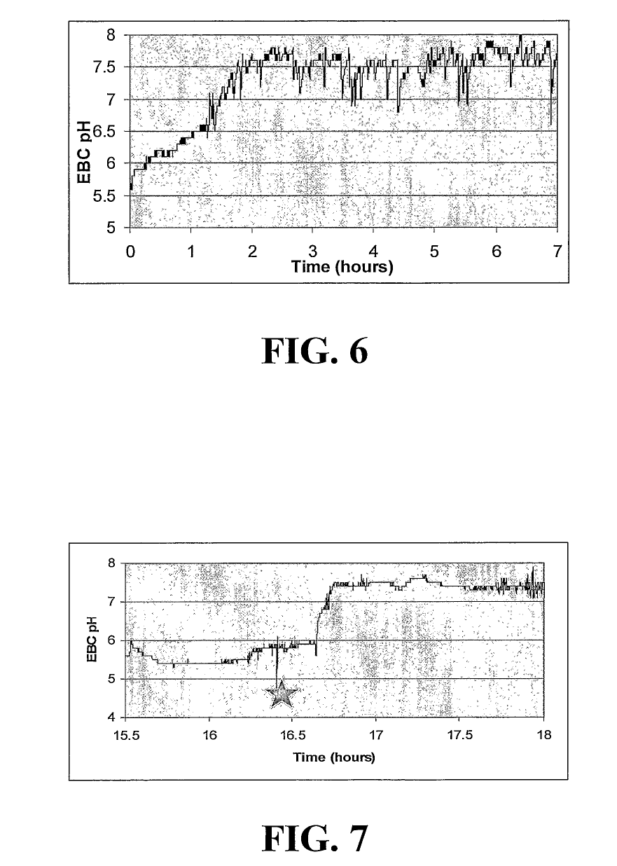

Referring to FIG. 6, FIG. 6 represents the graphical data sets captured from a 14 year old female with asthma requiring intubation and mechanical ventilation for profound obstructive airways following a seizure and possible acid aspiration event six hours before this EBC pH tracing was initiated. Arterial pH values as low as 6.7 were present during the previous six hours. The patient began clinically to improve approximately as this tracing was initiated. She improved sufficiently to be extubated at the seventh hour of this tracing. Note the gradual normalization of EBC pH over several hours prior to her improvement.

### Example No. 4

Referring to FIG. 7, FIG. 7 represents the graphical data sets captured from a cystic fibrosis patient with severe bronchiectasis and respiratory failure. Sodium bicarbonate was instilled into the endotracheal tube as a mucolytic agent (delineated by blue star). Within ten minutes, the airway alkalinizing effect of this therapeutic maneuver is readily noted by the continuous or continual EBC pH measurement system. Note that, because of the deaeration and removal of CO2, what is actually identified is the airway alkalinization and elimination of acid volatility.

### Example No. 5

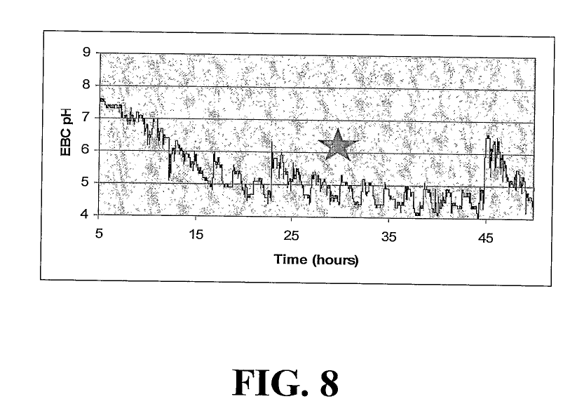

Referring to FIG. 8, FIG. 8 represents the graphical data sets captured from a 2-year-old intubated for tracheal reconstruction because of tracheal stenosis. At the time of initiating this EBC pH recording, the patient had been sedated post-operatively for seven days. The EBC pH tracing reveals a rapid decline over ten hours from her normal initial EBC pH. No clinical evidence of respiratory disease was present until more than 24 hours into the recording, at which point the patient had increased airway obstruction and prominent hypersecretion. Respiratory Syncytial Virus was diagnosed by PCR approximately 30 hours into the recording (red star). The EBC pH tracing predicted the event. Note also the extended sawtooth pattern. It can be speculated that the patient is aspirating enteral nutrition formula intermittently, and that her airway pH declined from the viral insult, transient pH upswings (toward the pH of the refluxed and aspirated formula) were able to be visualized. This patient was being treated with proton pump inhibitors to minimize stomach acidity.

### Example No. 6

In an embodiment, for example, the present invention method and system may be implemented that entails either an active or passive feedback system in which removal of EBC from the collection chamber(s) can occur at rates that depend on the volume of EBC forming and collecting. Additionally, if EBC collection slows below the rate of gas-standardization induced evaporation, the gas-standardization process is slowed or stopped to prevent complete evaporation (which can adversely affect the pH probe or other testing device), and a warning sound and visual signal may be noted on the electronic monitor and annotated in the electronically stored EBC pH database. Alternatively, specific chamber geometries may satisfactorily ensure probe wetting under all foreseen circumstances eliminating the need for an active control system.

Furthermore, the pace of gas-standardization and removal of EBC from the assay chamber must be sensitive to larger than average EBC accumulation rates, such as may occur when intubated subjects require high minute volumes to maintain sufficient ventilation. Excessive accumulation of EBC could possibly overwhelm the capacity to deaerate prior to measurement. Differing EBC accumulation rates, as discussed above, also would lead to a change in the moving average duration for any given volume of the pH assay chamber. The faster the accumulation of EBC, the shorter the duration of the moving average, such that it may be as low as a five-minute moving average. The shorter the duration, the more sensitive the system will be to short term changes in EBC pH, and so this is not to be avoided but rather encouraged. However, as the duration of the moving average shortens, it is necessary to assure that complete gas-standardization of the collected EBC is taking place.

A possible implementation will be to maintain the safety of the redundant EBC drainage system, such that there is no opportunity for EBC to accumulate to the point of interfering with exhalation flow.

The ability of the device to monitor EBC collection rate may be implemented by the system consisting of an optical proximity sensor (or other available type of sensor) connected to simple on/off controller logic, for example. This optical sensor will be positioned in a protected area with line of sight to the fluid pool such that the surface level of this pool can be seen. The logic circuit will control power to the coolant circuit, thus controlling condensation rate.

In essence the present invention system may monitor for excessively low EBC accumulation rates, disengage the gas-standardization system, stop pH recording (or other characteristic recording), and annotate an error on the visual screen and in the logged database. Furthermore, if EBC is accumulating too rapidly, the system will slow the EBC formation by modifying and/or intermittently shutting down the cooling system, slowing the flow of the cooling system, or increasing the temperature of the cooling system.

Development of methodology for gas-standardization in the absence of a facility capable of providing oxygen, nitrogen or oxygen tanks requires the development of a portable system for removing carbon dioxide from ambient air. carbon dioxide-free air is fully sufficient for gas-standardization of EBC, and allows for use of the continuous or continual EBC pH system where other sources of carbon dioxide-free gas might be in short supply. Short supplies of purified oxygen are unlikely to be encountered in medical facilities in the US, but are a conceivable occurrence in deployment situations, on ships, etc. Given the highly informative data one can achieve from monitoring EBC pH, provisions at assuring flexibility regarding gas-standardization may be implemented as well.

The present invention system may require a modest pressure compressed air source routed through a portable carbon dioxide scrubbing system. The output gas can be directly pumped into the deaeration chamber to produce the desired results.

Although the ability to gas-standardize with modified ambient air will be not be in demand in the civilian ICU's, there is another dual use motivation. Gas standardization currently requires a tank of some carbon dioxide-free gas. The new carbon dioxide scrubber would allow individual EBC pH samples collected from orally breathing subjects at work, school, etc, to be assayed on the spot. Further, with sufficient miniaturization, this process would allow for home EBC pH measurements. As home EBC collections are already being performed extensively by subjects in their own homes, the ability to measure their own EBC pH is applicable for the present invention as well.

The various embodiments of the present invention system and method as discussed throughout may be utilized for a variety of interfaces, functions, purposes, methods and systems including as discussed in the following patents, applications and publications—and may be utilized with the following patents, applications and publications—listed below and of which are hereby incorporated by reference herein in their entirety:

The present Application is also related to: U.S. Pat. No. 6,585,661 B1 issued Jul. 1, 2003, entitled “Device and Method for Monitoring Asthma;” U.S. Pat. No. 6,033,368 issued Mar. 7, 2000, entitled “Condensate Colorimetric Nitrogen Oxide Analyzer;” U.S. patent application Ser. No. 10/474,979, filed Oct. 16, 2003 (U.S. 2004/0127808 A1, published Jul. 1, 2004), entitled “Device and Method of Assessing Asthma and Other Diseases;” U.S. patent application Ser. No. 10/257,912 (U.S. 2003/0208132 A1 published Nov. 6, 2003), filed Oct. 17, 2002, entitled “Method and Device for Collecting and Analyzing Exhaled Breath;” U.S. Patent Application Publication No. 2004/0210151 A1 to Tsukashima et al. entitled “Respiratory Monitoring, Diagnostic and Therapeutic System;” and U.S. Patent Application Publication No. 2004/0210153 A1 to Tsukashima et al. entitled “Respiratory Monitoring, Diagnostic and Therapeutic System.”

One skilled in the art can appreciate that many other embodiments of condensate chamber and collection chambers, and other details of construction constitute non-inventive variations of the novel and insightful conceptual means, system and technique which underlie the present invention.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

We claim:

1. A method for monitoring a respiratory condition of a subject based on exhaled breath received from the subject, said method comprising:
continuously or continually collecting exhaled breath condensate (EBC) produced from the subject's exhaled breath; and
continually or continuously measuring one or more characteristics of the EBC.

2. The method of claim 1, further comprising gas standardizing the EBC.

3. The method of claim 2, wherein said gas standardizing comprises deaerating.

4. The method of claim 1 or 2 wherein the exhaled breath is received from the subject by way of a ventilator.

5. The method of claim 1 or 2 wherein the exhaled breath is received from the subject by way of a non-invasive respiratory device.

6. The method of claim 5, wherein said non-invasive respiratory device comprises a mask, mouthpiece, or other non-invasive breath collection or directing system.

7. The method of claim 5, wherein said non-invasive respiratory device comprises at least one of PAP device, CPAP device, or BiPAP device.

8. The method of claim 1 or 2 wherein the exhaled breath is received from the subject by way of an invasive respiratory device.

9. The method of claim 8, wherein said invasive respiratory device comprises at least one of endotracheal device, endotracheal tube or tracheostomy tube.

10. The method of claim 8, wherein said invasive respiratory device comprises a at least one of PAP device, CPAP device, IPPV device, SIMV device or BiPAP device, or mechanical machines used in intensive care units, operating rooms or at home.

11. The method of claim 2, comprising:
collecting the EBC in a first chamber, and wherein the gas standardizing comprises applying a first chamber gas supply to said collected EBC in said first chamber.

12. The method of claim 11, comprising:
collecting the EBC in a second chamber after traveling through said first chamber.

13. The method of claim 12, wherein the gas standardizing further comprises applying a second chamber gas supply to said collected EBC in said second chamber.

14. The method of claim 13, further comprising:
controlling the flow of applied second chamber gas supply to said second chamber.

15. The method of claim 14, further comprising:
controlling the flow of applied first chamber gas supply to said first chamber.

16. The method of claims 14, wherein said continual or continual measurement of one or more characteristics of the EBC comprises using a means for testing the EBC to determine the one or more characteristics of the EBC, said testing means being in fluid communication with EBC in said first and/or second chamber.

17. The method of claim 11, further comprising:
controlling the flow of applied first chamber gas supply to said first chamber.

18. The method of claims 1 or 2, wherein said continual or continuous measurement of one or more characteristics of the EBC comprises using a means for testing the EBC to determine the one or more characteristics of the EBC.

19. The method of claim 18, wherein said testing means comprises at least one of electronic monitor, monitor, probe, or electric probe, or any combination thereof.

20. The method of claim 18, wherein the one or more characteristics of the EBC comprises pH.

21. The method of claim 18, wherein the one or more characteristics of the EBC comprises at least one of oxidation/reduction potential (ORP), ammonia, conductivity, a specific anion or cation probe, nitrogen oxides or redox potential.

22. The method of claim 18, further comprising:
acquiring data from said testing means.

23. The method of claim 22, further comprising:
evaluating the acquired data.

24. The method of claim 23, wherein said evaluation comprises:
determining the presence, absence or status of a disease or response to therapy.

25. The method of claim 24, wherein said disease is a respiratory disease.

26. The method of claim 23, wherein said evaluation comprises determining presence, absence or status of at least one of the following: asthma, chronic obstructive pulmonary disease (COPD), ventilator-associated pneumonia (VAP), gastric acid reflux and aspiration, respiratory viral infections (including rhinovirus, respiratory syncitial virus, and others) chemical exposures (such as chlorine gas), near-drownings, acute respiratory distress syndrome (ARDS), acute lung injury, trauma (chest, multi-organ or other).

27. The method of claim 1, wherein said collect EBC collects in a first chamber.

28. The method of claim 27, wherein said collected EBC additionally collects in a second chamber.

29. The method of claim 1, comprising cooling the exhaled breath to provide the collected EBC.

30. The method of claims 1 or 2, further comprising:
evaluating the acquired data.

31. The method of claim 30, wherein said evaluation comprises:
determining the presence, absence or status of a disease or response to therapy.

32. The method of claim 31, wherein said disease is a respiratory disease.

33. The method of claim 30, wherein said evaluation comprises determining presence, absence or status of at least one of the following: asthma, chronic obstructive pulmonary disease (COPD), ventilator-associated pneumonia (VAP), gastric acid reflux and aspiration, respiratory viral infections (including rhinovirus, respiratory syncitial virus, and others) chemical exposures (such as chlorine gas), near-drownings, acute respiratory distress syndrome (ARDS), acute lung injury, trauma (chest, multi-organ or other).

34. The method of claim 1, wherein duration of said continuous or continual collection is equal to a single exhaled breath or a portion of a single exhaled breath from the subject.

35. The method of claim 1, wherein duration of said continuous or continual collection is greater than a single exhaled breath from the subject.

36. The method of claim 1, wherein duration of said continual or continuous measurement is at least as great as a period sufficient to acquire data required for said measurement.

37. The method of claim 36, wherein said acquisition is accomplished using an electronic, electrical or analog device or any combination thereof.

38. The method of claim 1, wherein duration of said continuous or continual collection is equal to the duration or portion of duration that is required for treatment, therapy, monitoring, test or experiment with the subject.

39. The method of claim 1, wherein duration of said continuous or continual collection is equal to a period of less than about 1 minute.

40. The method of claim 1, wherein duration of said continuous or continual collection is equal to a period of about 0.5 minutes to about 5 minutes.

41. The method of claim 1, wherein duration of said continuous or continual collection is equal to a period of about 1 minute to about 1 hour.

42. The method of claim 1, wherein duration of said continuous or continual collection is equal to a period of about 1 hour to about 12 hours.

43. The method of claim 1, wherein duration of said continuous or continual collection is equal to a period of about 12 hours to about a day.

44. The method of claim 1, wherein duration of said continuous or continual collection is equal to a period of about 1 day to about 1 year.

45. The method of claim 1, wherein duration of said continuous or continual collection is equal to a period greater than about 1 year.

46. The method of any one of claims 34-45, wherein at least one interruption occurs during period of said collection.

47. A method for monitoring a respiratory condition of a subject based on exhaled breath received from the subject, said method comprising:
continuously or continually collecting exhaled breath condensate (EBC) produced from the subject's exhaled breath; and
continually or continuously measuring PH of the EBC.

48. The method of claim 47, further comprising:
evaluating the acquired data.

49. The method of claim 48, wherein said evaluation comprises:
determining the presence, absence or status of a disease or response to therapy.

50. The method of claim 49, wherein said disease is a respiratory disease.

51. The method of claim 48, wherein said evaluation comprises determining presence, absence or status of at least one of the following: asthma, chronic obstructive pulmonary disease (COPD), ventilator-associated pneumonia (VAP), gastric acid reflux and aspiration, respiratory viral infections (including rhinovirus, respiratory syncitial virus, and others) chemical exposures (such as chlorine gas), near-drownings, acute respiratory distress syndrome (ARDS), acute lung injury, trauma (chest, multi-organ or other).

52. A system for monitoring a respiratory condition of a subject, said system comprising:
an exhaled breath port in communication with a condensate chamber; said condensate chamber being configured to condense the subject's exhaled breath received from said port and produce exhaled breath condensate (EBC);
a first collection chamber configured to continuously or continually collect the EBC;
a drain off port configured to drain the collected EBC in said first collection chamber as the collected EBC reaches a predetermined volume; and
a means for continually or continuously measuring one or more characteristics of the collected EBC.

53. The system of claim 52, further comprising
a gas standardizing means for standardizing the collected EBC from the subject, said gas standardizing means being in communication with said collected EBC.

54. The system of claim 53, wherein said gas standardizing comprises a gas supply.

55. The system of claim 54, wherein said gas supply comprises at least one of oxygen, argon, or nitrogen or any gas mixture from which carbon dioxide has been scrubbed or removed by available means.

56. The system of claim 52, wherein said continual or continuous measurement of one or more characteristics of the EBC comprises a means for testing the EBC to determine the one or more characteristics of the EBC, said testing means being in fluid communication with EBC in said first chamber.

57. The system of claim 52, comprising a cooling device in communication with said condensate chamber to produce the EBC.

58. The system of claim 57, wherein said cooling device comprises at least one of cooling sleeve, ice, endothermal chemical reaction, cold water bath, thermocouple cooling technique, heat pump, heat sink or other available chilling process/device.

59. The system of claim 52, wherein duration of said continuous or continual collection is equal to a single exhaled breath or a portion of a single exhaled breath from the subject.

60. The system of claim 52, wherein duration of said continuous or continual collection is greater than a single exhaled breath from the subject.

61. The system of claim 52, wherein duration of said continual or continuous measurement is at least as great as a period sufficient to acquire data required for said measurement.

62. The system of claim 61, wherein said acquisition is accomplished using an electronic, electrical or analog device or any combination thereof.

63. The system of claim 52, wherein duration of said continuous or continual collection is equal to the duration or portion of duration that is required for treatment, therapy, monitoring, test or experiment with the subject.

64. The system of claim 52, wherein duration of said continuous or continual collection is equal to a period of less than about 1 minute.

65. The system of claim 52, wherein duration of said continuous or continual collection is equal to a period of about 0.5 minutes to about 5 minutes.

66. The system of claim 52, wherein duration of said continuous or continual collection is equal to a period of about 1 minute to about 1 hour.

67. The system of claim 52, wherein duration of said continuous or continual collection is equal to a period of about 1 hour to about 12 hours.

68. The system of claim 52, wherein duration of said continuous or continual collection is equal to a period of about 12 hours to about a day.

69. The system of claim 52, wherein duration of said continuous or continual collection is equal to a period of about 1 day to about 1 year.

70. The system of claim 52, wherein duration of said continuous or continual collection is equal to a period greater than about 1 year.

71. The system of any one of claims 59-70, wherein at least one interruption occurs during period of said collection.

72. The system of claims 52 or 53, further comprising:
a means for evaluating the acquired data.

73. The system of claim 72, wherein said evaluation comprises:
determining the presence, absence or status of a disease or response to therapy.

74. The system of claim 73, wherein said disease is a respiratory disease.

75. The system of claim 72, wherein said evaluation comprises determining presence, absence or status of at least one of the following: asthma, chronic obstructive pulmonary disease (COPD), ventilator-associated pneumonia (VAP), gastric acid reflux and aspiration, respiratory viral infections (including rhinovirus, respiratory syncitial virus, and others) chemical exposures (such as chlorine gas), near-drownings, acute respiratory distress syndrome (ARDS), acute lung injury, trauma (chest, multi-organ or other).

76. A system for monitoring a respiratory condition of a subject, said system comprising:
an exhaled breath port in communication with a condensate chamber; said condensate chamber being configured to condense the subject's exhaled breath received from said port and produce exhaled breath condensate (EBC);
a first collection chamber configured to continuously collect the EBC;
a second collection chamber in fluid communication with said first collection chamber and configured to continuously or continually collect the EBC from the first collection chamber as the first chamber collected EBC reaches a predetermined level in said first collection chamber;
a drain off channel configured to drain the collected EBC in said second collection chamber as the collected EBC in said second collection chamber reaches a predetermined volume; and
a means for continually or continuously measuring one or more characteristics of the collected EBC.

77. The system of claim 76, comprising:
an inter-chamber port configured to pass the collected EBC in said first chamber to said second chamber.

78. The system of claim 76, further comprising
a gas standardizing means for standardizing the collected EBC from the patient, said gas standardizing means being in communication with said collected EBC in said first and/or second collection chamber.

79. The system of claim 78, wherein said gas standardizing comprises a gas supply.

80. The system of claim 79, wherein said gas supply comprises at least one of oxygen, argon, or nitrogen or any gas mixture from which carbon dioxide has been scrubbed or removed by available means.

81. The system of claim 76, comprising a cooling device in communication with said condensate chamber to produce the EBC.

82. The system of claim 81, wherein said cooling device comprises at least one of cooling sleeve, ice, endothermal chemical reaction, cold water bath, thermocouple cooling technique, heat pump, heat sink or other available chilling process/device.

83. The system of claim 76, wherein duration of said continuous or continual collection is equal to a single exhaled breath or a portion of a single exhaled breath from the subject.

84. The system of claim 76, wherein duration of said continuous or continual collection is greater than a single exhaled breath from the subject.

85. The system of claim 76, wherein duration of said continual or continuous measurement is at least as great as a period sufficient to acquire data required for said measurement.

86. The system of claim 85, wherein said acquisition is accomplished using an electronic, electrical or analog device or any combination thereof.

87. The system of claim 76, wherein duration of said continuous or continual collection is equal to the duration or portion of duration that is required for treatment, therapy, monitoring, test or experiment with the subject.

88. The system of claim 76, wherein duration of said continuous or continual collection is equal to a period of less than about 1 minute.

89. The system of claim 76, wherein duration of said continuous or continual collection is equal to a period of about 0.5 minutes to about 5 minutes.

90. The system of claim 76, wherein duration of said continuous or continual collection is equal to a period of about 1 minute to about 1 hour.

91. The system of claim 76, wherein duration of said continuous or continual collection is equal to a period of about 1 hour to about 12 hours.

92. The system of claim 76, wherein duration of said continuous or continual collection is equal to a period of about 12 hours to about a day.

93. The system of claim 76, wherein duration of said continuous or continual collection is equal to a period of about 1 day to about 1 year.

94. The system of claim 76, wherein duration of said continuous or continual collection is equal to a period greater than about 1 year.

95. The system of any one of claims 83-94, wherein at least one interruption occurs during period of said collection.

96. The system of claims 76 or 78, further comprising:
a means for evaluating the acquired data.

97. The system of claim 96, wherein said evaluation comprises:
determining the presence, absence or status of a disease or response to therapy.

98. The system of claim 97, wherein said disease is a respiratory disease.

99. The system of claim 96, wherein said evaluation comprises determining presence, absence or status of at least one of the following: asthma, chronic obstructive pulmonary disease (COPD), ventilator-associated pneumonia (VAP), gastric acid reflux and aspiration, respiratory viral infections (including rhinovirus, respiratory syncitial virus, and others) chemical exposures (such as chlorine gas), near-drownings, acute respiratory distress syndrome (ARDS), acute lung injury, trauma (chest, multi-organ or other).
